(19) United States
(12) Patent Application Publication
(10) Pub. No.: US 2017/0022267 A1
(43) Pub. Date: Jan. 26, 2017

(54) METHODS FOR SYSTEMATIC CONTROL OF PROTEIN STABILITY

(71) Applicant: UChicago Argonne LLC, Chicago, IL (US)

(72) Inventor: Fred J. Stevens, Naperville, IL (US)

(73) Assignee: UChicago Argonne LLC, Chicago, IL (US)

(21) Appl. No.: 15/287,146

(22) Filed: Oct. 6, 2016

Related U.S. Application Data
(62) Division of application No. 13/002,013, filed on Feb. 10, 2011, now abandoned.
(60) Provisional application No. 61/080,563, filed on Jul. 14, 2008.
(60) Provisional application No. 61/150,562, filed on Feb. 6, 2009.

(51) Int. Cl. C07K 16/18; C07K 16/00

(52) U.S. Cl. CPC C07K 16/18; C07K 16/00; C07K 2317/94; C07K 2317/34; C07K 2317/92; C07K 2317/515; C07K 2317/622

(57) ABSTRACT

Methods and compositions to control the stability of proteins with special emphasis on antibodies and proteins with antibody-like structures, e.g., having an “immunoglobulin-like” fold, are described. Controlling the stability facilitates different applications for a protein with the same function, but different stability.

### CROSS-REFERENCE TO RELATED PATENT APPLICATIONS

This application claims priority to U.S. Provisional Application No. 61/080,563, filed Jul. 14, 2008, and 61/150,562, filed Feb. 6, 2009, the contents of which applications are incorporated herein by reference in their entireties.

The United States Government has rights in this invention pursuant to Contract No. DE-AC02-06CH11357 between the U.S. Department of Energy and UChicago Argonne, LLC, operator of Argonne National Laboratory.

### BACKGROUND

Methods and compositions to control the stability of proteins, with special emphasis on antibodies and proteins with antibody-like structures, e.g., having an “immunoglobulin-like” fold, are described. Controlling the stability of the proteins facilitates different applications for proteins that have the same function, but different stabilities.

Protein instability reduces shelf life due to changes in folding, resulting in altered or loss of function. Stabilization of antibodies, or any other protein, has been traditionally a trial-and-error process, potentially time-consuming and expensive, with little assurance of success. Theoretically, a polypeptide of N amino acids may exhibit 19^{N} alternative amino acid sequences. Most of these sequences do not produce a functional antibody or other protein, and little insight has been developed to minimize the experimental effort to test the large numbers of amino acid replacements that must be experimentally addressed in a “brute force” method in order to control stability. A structure determined by x-ray analysis of a crystallized protein is not necessarily an accurate representation of the protein in solution. For example, the most common atom in a protein, hydrogen, is invisible to x-ray analysis. Computational analysis cannot reliably optimize the stability of a protein.

Antibodies are protein molecules that are produced by higher organisms. They include “light” and “heavy” chains. Antibodies are the basis of the adaptive immune system, which provides a natural response against infection by viruses, bacteria, and fungi. Antibodies can be evoked by vaccines, resulting in immunization against diseases such as polio. Similarly, antisera that contain antibodies that recognize particular molecules of interest can be generated by innoculating animals with molecules for which a detection method is desired. This capability is the basis of the multibillion dollar immunodiagnostics industry and the emerging immunotherapeutics field that provides treatments for diseases such as rheumatoid arthritis and some cancers.

Antibodies are widely used in therapeutic, diagnostic, imaging, bioremediation, sensor, and research applications. Antibodies have been very successful particularly in therapeutic applications. The US Food and Drug Administration (FDA) has approved 23 antibodies (see Table 4 for a list of representative antibodies) and about 200 antibodies are in clinical development. The global market for antibody therapeutics was estimated at $25 billion (2007). The market is expected to reach $45 billion by 2012. Eight antibodies have reached a blockbuster level of sale defined as $1 billion or more in annual sales revenue (highlighted in Table 4). Antibody therapeutics is one of the fastest growing sectors in the pharmaceutical industry. Average annual growth rate (AAGR) for the antibody therapeutic market is 11.5%.

Antibodies are the ultimate example of combinatorial biochemistry. Each human is thought to be capable of producing on the order of one billion different antibodies, generating a library that exceeds the diversity of any that has been produced by combinatorial chemistry efforts. The binding site of an antibody is formed at the junction of two protein domains or modules. Thus, different combinations of these domains lead to different combinations of amino acid residues in the binding site. Different patterns of amino acids result in different binding specificity.

Antibodies are made up of several relatively small beta-sandwich domains that exhibit a structure termed the immunoglobulin fold. Examples of other well-known proteins that share this fold, and may have an evolutionary link to antibodies, include tumor necrosis factor, Cu, Zn—superoxide dismutase, and transthyretin. Antibodies generally function by generating a binding site from the juxtaposition of two variable domains, one from the light chain and one from the heavy chain. The modules that make up the binding sites of antibodies are known as variable domains. “Variable” indicates differences in the amino acid sequences generated by the several genes that provide alternative amino acid sequences for each module. One of the modules is known as the heavy chain variable domain and the other as the light chain variable domain, referring to the two types of polypeptide chains from which antibodies are assembled. The light chain consists of one variable domain at the starting point of the protein followed by one constant domain. The heavy chain consists of one variable domain at the starting point of the antibody followed by three or four constant domains.

The constant domains are so termed because they exhibit little amino acid variation in contrast to variable domains that have highly diverse primary structures (amino acid sequences).

Variability of primary structures arises from several sources including (1) most antibody producing animals contain multiple versions of genes for light chain and heavy chain variable domains, and (2) the cells that produce antibodies are programmed to be very error-prone during early stages of replication, leading to high rates of somatic mutation. One consequence of somatic mutation is diversity of antibody specificities. Another consequence of somatic mutation is loss of stability; i.e., decreased tolerance to temperature or other factors leading to increased rate of loss of function.

There are similarities in all mammalian immune systems. In the mouse, approximately 100 light chain variable domains can be combined with more than 200 heavy chain domains.

Humans have at least 50 and 40 light and heavy chain variable domain genes, respectively. This basic set would yield only 2000 different combinations or 2000 different binding sites. However, as the cells that produce antibodies mature, additional mechanisms, largely mutations, result in several billion different combinations. Many of these potential binding sites are filtered out of the collection if they react with molecules in the body.

Monoclonal antibodies are generally considered to be monospecific antibodies in the sense that they are identical because they are produced by one type of immune cell from clones of a single parent cell. To generate a monoclonal antibody a clone of cells is prepared, all of which produce the same antibody. A method to accomplish this is to first immunize mice with an antigen of interest (the “target antigen”). After some time, a large number of cells that produce antibodies that bind to the target antigen can be found in the spleen of the mouse. When spleen cells are fused to cells of an antibody-producing type of laboratory cancer cell line, some hybrid cells result that yield the antibody of interest and that can grow and divide indefinitely (“immortal”). Thus, monoclonal antibodies can be produced against essentially any antigen.

Another contemporary strategy for acquiring antibody-type reagents is to collect, usually from human antibody-producing cells, the pieces of RNA that contain the information for the amino acid sequences of light and heavy chain variable domains. The RNA is used to generate complementary DNA (cDNA), These pieces of light and heavy chain cDNA are linked together and inserted into the gene for a protein that is exposed on the surface of a virus that attacks bacteria. This results in a library of viruses known as phage that exhibit or display a large number of different combinations of light and heavy chain variable domains on their surfaces. When exposed to immobilized target molecules, some of the viruses are likely to bind to the target. When removed from the target molecules, and used to infect bacteria, a large quantity of viruses that generate antibody-type particles result. In principle, the DNA that encodes the antibody-type particle can be transferred to E. coli, and the antibody (protein) is produced by the bacteria. In practice, this procedure often results in a very unstable construct that is not useful. As a consequence, the tremendous potential of this technology, known as phage display, to produce scFv constructs (single chain antibody variable fragments), cannot be achieved if the instability problem is not resolved.

scFv constructs are inherently unstable due to a large surface to volume ratio and the use of a long flexible linker to join the VH and VL domains. In fact, the stability of all antibodies is limited due to the lack of evolutionary pressure to push stability beyond a physiologically useful average. The potential benefits of antibodies with above average stability include: improved productivity of a research and development pipeline, i.e. more successes and a simplified formulation, leading to lowered cost of antibody production whether for therapeutics, diagnostics, biosensors, or other applications that are only possible with stabilized antibodies. More stable antibodies with longer shelf-life also result in enhanced patient safety and minimize waste due to expiration of products. Finally, the use of stable antibodies permits the development of novel immunotherapeutics strategies.

Stability may be measured in terms of thermodynamic equilibrium or by tolerance of elevated temperature, pH variation, or other challenges. The term “stability” refers to the ability of a protein to maintain its native conformation and function in response to changes in environmental factors such as temperature, pH, and ionic strength.

The average serum half-life of natural antibodies (IgG) is 23 days. Most of the commercially available antibodies have a much lower half-life (see Table 5 for representative examples). Stability appears to be compromised during antibody engineering. Stability is important at every step: manufacturing, storage, formulation, shipping, dosing, and pharmacokinetic. There have been numerous and costly failures over the past 15 years because stability was not always considered a key issue.

Due to concern for stability, antibodies require refrigeration for long-term preservation; this limits the application of antibodies to controlled environments. A conventional approach to increase stability is random mutagenesis in which a gene coding for an antibody is subjected to random mutagenesis to generate a library of hundreds (or even thousands) of mutants each of which is tested for stability. The method is costly, time consuming, and highly unpredictable. Besides random mutagenesis, one can roughly classify previous approaches into three categories: (1) domain-specific alteration, (2) “directed” evolution; and (3) high-throughput brute force.

Domain Specific Alteration.

This category constitutes the majority of the literature relevant to antibody stabilization. It does not represent a strategy for systematic stabilization of antibodies, but rather is a compilation of various modifications that are useful in specific cases. For instance, replacement of methionine at position 4 with leucine in kappa light chains was reported to result in improved stability, presumably due to a smaller entropy penalty from immobilizing the shorter leucine side chain. Another example focused on three amino acids in the heavy chain variable domain.

Another strategy involves transplanting loops from an antibody of desired specificity into a different antibody framework of appropriate stability, a strategy that may have success if the corresponding antibody domain fragments have precisely the same conformation and if amino acids in the transferred loops are not responsible for loss of stability. However, amino acids responsible for specificity and high affinity are usually introduced by mutation and are frequently destabilizing.

In some cases, it is possible to improve folding by modifying amino acids at selected positions in turns between beta strands in the domains. However, this approach has not been generalized. Consensus statistics identifying the most commonly found amino acid at particular positions have been reported as a useful guide for stabilization. This is valid to the extent that it restores divergence from the germline sequence, which can often improve stability but can also have the opposite effect. However, as noted in a preceding paragraph, the consensus methionine at position 4 in the kappa variable domains is destabilizing compared to seldomly observed leucine. The consensus approach, while useful, is restricted by the fact that the structure of antibodies did not evolve to have maximum stability, but only a sufficient level of stability.

Each of the examples cited above provides a possible enhancement to affinity on a case-by-case basis. In toto, however, these methods do not provide a basis for asserting that, with them, any antibody can be significantly improved in stability.

Directed Evolution.

Conventionally, the designation of “directed evolution” has been applied to approaches in which an enzyme critical for the survival of microorganism is subjected to mutation and stress challenges so that surviving cells are those that have more robust forms of the enzyme. Since antibodies are not critical for the survival of bacteria, this designation is a loose description. In this instance, its justification is based on subjecting phage display libraries of single chain variable domain binding fragments (scFvs) to harsh conditions. An exception is using error prone PCR to construct a library of variants of a prion-binding antibody, resulting in the selection of an antibody with picomolar affinity. Effectively however, the general approach culls the phage display library of unstable scFv constructs. Concurrently, it diminishes the diversity of the library, thus reducing the probability of being able to capture antibody constructs of useful specificity and affinity. What is needed is an approach that maximizes the probability of identifying antibodies of utility, with stabilization implemented as a second step.

High-Throughput Brute Force.

The availability of robotics to a growing number of molecular biology laboratories has enabled large scale screening of the effects of a large number of site-specific mutations. A system is reported to improve the stability, and expression levels, of an Fab. The key element of this approach was to analyze the database of variable domain primary structures to identify positions of high variability. Automated methods for sequence alignment are used and variability is assessed as Shannon's entropy, a metric derived from information theory. Positions of highest entropy are reported to be most tolerant of amino acid changes. Identifying 45 positions in the Fab, robotic methods and saturation mutagenesis were used to construct variants for evaluation, also undertaken by robotics. Saturation mutagenesis led to the construction and evaluation of more than 850 mutants. Obviously, the method is very laborious, and every future Fab stabilization project will require the same level of effort.

Although, on the surface, this approach seems reasonable, there would appear to be several flaws. The inference that positions of highest entropy are tolerant of amino acid changes is reasonable; the suggestion that these are the optimal positions to screen for stabilizing changes is less so. Tolerance of amino acid change implies that the changes are of little consequence, and are unlikely to contribute significantly to stability. Moreover, most of the positions of high variability are located in the complementarity determining regions; thus, most of the amino acids variations introduced by this strategy are at positions that could affect binding properties. In addition, the database contains many “nonsensical” sequences; i.e., mRNA-based sequences that incorporate frameshifts generating artificial variability. Automated methods of data gathering are unlikely to filter them out. Finally, automated methods will create artificial variability at the edges of complementarity determining regions due to inconsistent positioning of insertion/deletions.

Unfortunately, each of the three methods described in the previous paragraphs is costly, time consuming and unpredictable.

An approach to the problem of instability is to provide suitable formulation excipients and other formulation conditions. However, many antibodies are inherently unstable and do not yield stable formulation despite the use of excipients. Some micro-organisms thrive at temperatures above the boiling point of water. Therefore, the instability of animal proteins is not due to an inviolable law of physics—the polymer is stable, the fold is not.

### SUMMARY

An approach to controlling protein stability described herein is to modify the amino acid sequence of the polymers that make up proteins such as antibodies to optimize stability for particular applications. A method is provided for identifying amino acids, which when substituted in target proteins, control the stability of the protein molecules resulting in, for instance, change in their shelf life and/or half-life. This method is particularly useful when the proteins have an immunoglobulin-like fold, e.g., antibodies. Use of the method results in engineered proteins with controllable stability.

A method for controlling the stability of a target protein molecule to a desired level, the method including:

(a) compiling databases of amino acid sequences of the proteins from man, mouse, and other animals to identify positions of no amino acid variation, high amino acid variation, and intermediate variation;

(b) replacing selected amino acid residues in the target protein molecule with compatible amino acids observed at that position in the relevant database to obtain a substituted protein molecule;

(c) determining stability and function of the substituted protein molecule;

(d) comparing the stability of the substituted protein molecule to the target protein molecule to determine if stability is controlled and there are no negative consequences on its functions;

(e) repeating steps (a)-(d) until the desired level of stability is achieved.

The protein molecule may be an antibody. A method of controlling the stability of a target antibody molecule to a desired level includes the steps of:

(a) compiling databases of amino acid sequences of the antibody variable domains of man, mouse, and other animals;

(b) postulating that the absence or near-absence (<1%) of certain amino acids at particular positions is due to incompatibility with production of a functional variable domain and that the presence of such amino acids at the position has been eliminated by evolutionary selection or by the quality control processes of the immune system;

(c) replacing selected amino acid residues in the target antibody molecule with compatible amino acids observed at that position in the relevant database to obtain a substituted antibody molecule;

(d) determining stability and function of the substituted antibody molecule;

(e) comparing the stability of the substituted antibody molecule to the target antibody molecule to determine if stability is controlled and there are no negative consequences on its function;

(f) repeating steps (a)-(e) until the desired level of stability is achieved.

Controlling includes enhancing stability while preserving function. The amino acids replaced may be in the variable chains of the antibody. Replacing amino acids may be done by site specific mutagenesis. A protein may be produced in bacteria, yeast, plant or animal cells. Enhanced stability may facilitate therapeutic, diagnostic and other uses of the protein.

Stability may be measured in terms of thermodynamic equilibrium or by tolerance of elevated temperature, pH variation, or other challenges. The term “stability” refers to the ability of a protein to maintain its native conformation and function in response to changes in environmental factors such as temperature, pH, and ionic strength.

### BRIEF DESCRIPTION OF THE DRAWINGS

FIG. 1 shows antibody utility as a function of stability and affinity. AG is change in free energy upon folding; K is affinity constant for the interaction between an antibody and its cognate antigen; “useful” means having sufficient affinity and stability for use in manufacture, formulation, storage, and the like, in therapeutic and diagnostic applications.

FIG. 2 shows multiple alignment (SEQ ID NOS: 60-64, respectively, in order of appearance) of human κ-4 amyloid light chains and improvement in stability by amino acid replacement.

FIG. 3 shows successful increase in thermodynamic stability of an amyloid light chain. The proportion of unfolded form of the light chain variable domain was monitored by the increase in fluorescence that occurred when unfolding allowed a normally buried tryptophan residue to have contact with water molecules in the solvent. The improved robustness of the “hyperstable” form of the variable domain results in a one-billion fold improvement in the ratio of native form to unfolded form when compared to the “unstable” variant that corresponds to an amyloid-forming light chain variable domain.

FIG. 4 shows increase in thermal stability of an amyloid light chain as measured by the enhanced fluorescence of an added dye when access to the hydrophobic core of the protein is made possible by unfolding. The curve to the far left is that obtained by an unstable variable domain corresponding to an amyloidogenic light chain; as seen, unfolding occurs at temperatures below 35° C. indicating that this protein was unstable at physiological body temperature. In contrast, the curve on the far right does not show observable unfolding until a temperature of 70° C. (˜160° F.) is achieved, in substantial excess of physiological temperatures. The control (non-pathological) protein variant (filled circles and open triangles) exhibited unfolding at approximately 47° C. (117° F.), also significantly above temperatures obtained physiologically. Other curves represent variable domain constructs that incorporate one or more amino acid variations found in amyloidogenic kappa-4 light chains.

FIG. 5 shows correlation between thermal (Tₘ) and thermodynamic (Cₘ) stability of antibodies.

FIG. 6 shows that increased stability of anti-laminin antibodies is not necessarily accompanied by significant decrease in binding to laminin. The curves represent binding of three variant anti-laminin scFv constructs to the sensor of a Biacore instrument. Laminin was adsorbed to sensor; antibody constructs were added at 0 seconds. Increased response indicates binding of antibody to laminin. As seen, the initial response was identical for all three antibodies; wild-type, 15-9; a mutant with glutamine replaced by alanine at position 55 (Q55A), and a mutant with aspartic acid replaced by asparagine at position 70 (D70N). Excess buffer was added 75 seconds; the decrease in response units corresponds to dissociation of the laminin-antibody complex. As observed, the D70N mutant exhibits the same dissociation rate as observed for the original antibody. The Q55A mutant displays the same or slightly slower dissociation rate. The affinity constant of the antibodies is determined by the ratio of binding rate to dissociation rate. Therefore, the affinities of the two stabilized mutants are at least equivalent to that of the original antibody.

FIG. 7 demonstrates that increased thermal and thermodynamic stability enhances resistance to a protease. LANE 1=mol wt standards, LANE 2=VH2 wild type. LANE 3=VH2-6, LANE 4=VH2-15.

FIG. 8 shows structural similarity between cupredoxins (Azurin) and antibody variable domains (VL).

FIG. 9 depicts a structure of Factor VIII that consists of 6 cupredoxin (ceruloplasmin) domains and two lactadherin-like domains at the C terminus. The central region is uncharacterized.

### DETAILED DESCRIPTION

A method is described for controlling the stability of proteins generally and of proteins with an antibody like structure (e.g., having “immunoglobulin-like” fold) specifically. Controlling the stability facilitates different applications of a protein with the same function, e.g., a long half-life is desirable for a therapeutic antibody, but a shorter half-life is desirable for certain applications such as radiotherapy or imaging. An aspect of the methods and compositions described herein is that multiple products may emerge from one antibody as a result of this invention. A very short half-life may be desirable for specific cases, for example, to prevent blood clotting during an emergency while allowing reasonably rapid restoration of clotting ability.

There are numerous advantages of using antibodies with increased stability. Highly stable antibodies generate less aggregates, are less prone to degradation and precipitation leading to increased yield and thus lowering the cost of production. Stable antibodies also allow liquid formulation thus avoiding lyophilization. Liquid formulation is preferred over lyophilized formulation as it is easy to administer and less expensive to manufacture. However, about half of the currently marketed antibodies are provided in lyophilized formulation as they are not stable enough to be formulated in liquid form. Lyophilization can denature antibodies to varying extent. Moreover, there is a varying degree of loss upon reconstitution of lyophilized antibodies as a result of aggregation and precipitation. Increased stability is expected to impart longer shelf life and longer serum half-life, the latter would allow decreased dosage and lower frequency of administration. This in turn would not only result in reduced side effects but will also lower the cost of treatment. Furthermore, stabilized antibodies can withstand much higher temperature, and therefore are suitable for applications in field (such as sensor to detect chemicals, explosives, and infectious agents) where the ambient temperature could be high.

The method described herein allows fine tuning of stability of antibodies in order to generate multiple products from the same antibody for different applications (see Table 6). For instance, most therapeutic applications require antibodies of medium half-life. On the other hand, antibodies with short half-life are preferred for applications such as imaging, radiotherapy, and certain therapy of short duration where it is desirable to use antibodies that are cleared rapidly from the body. Diagnostic and biosensor applications, on the other hand, would require antibodies with significantly higher stability. Therefore, once a high affinity antibody is identified and characterized, the method described herein could be used to derive multiple forms of the same antibody differing only in the level of stability that are suitable for different applications.

Identification of a protein's fold (three-dimensional structure) provides information revealing the 3D organization of its secondary structural elements, but does not necessarily provide the overall detailed description of the tertiary structure that may ultimately be needed to explain the properties of the protein. Nevertheless, recognition from the primary structure of a new protein that it shares a fold with other proteins of known structure and function enables detailed examination of the sequence for the particular amino acids that have been identified as structural determinants of function including interactions with protein partners and binding of small molecule ligands. Therefore, there is a direct benefit from improvements in the ability to recognize fold from sequences in that such recognition can directly lead to hypotheses to define function. The three-dimensional structures of many antibody variable domains are known, providing extra guidance for generation of the amino acid replacement candidates. More primary structure data is available for light and heavy chain variable domains than for any other protein family. This is because of the existence of a database consisting of the amino acid sequences of variable domains produced by patients with cancers of antibody-producing cells (myeloma) and for which pathological properties correlate with stability. All amino acid changes characterized experimentally during the stabilization process for one antibody will contribute to varying degrees to the stabilization of all subsequent antibodies. This is because all molecules of this type have particular amino acid residues at the exact same positions along the peptide roster, as they must, such that every VL can assemble with every VH within the same species. Antibodies in any configuration are suitable for the stability-enhancing method, including antibody fragments such as single variable domains. Fv and scFv constructs, Fabs, and whole antibodies, e.g. anti-botulinum toxin and anti-anthrax spores.

The term “antibody” is used herein in the broadest sense and specifically includes full-length antibodies, antibody fragments, chimeric antibodies, humanized antibodies, and human antibodies. “Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments.

Stability of a protein fold is determined by the sum of various interactions among the amino acid side chains (e.g., hydrogen bonding, and electrostatic, van der Waal, and hydrophobic interactions) that preserve the functional fold and the entropy change during folding. Stability can be improved by increasing the number of favorable interactions and/or removing unfavorable interactions. Such improved stability enables the use of what may be termed an inverse structure activity relationship (SAR) strategy to improve antibody affinity.

The SAR approach is a common practice in the pharmaceutical industry. Typically, following identification of a drug candidate, chemical features are systematically varied. Some of these variations improve binding characteristics of the drug for its physiological target. These chemical changes are then combined to generate an optimized drug. In the case of improving binding properties of an antibody, amino acid changes are made one at a time, at positions that are likely to make contact with the target molecule. Changes involve variations in chemistry; charge, hydrophobicity and size. Some of these changes may improve binding properties and can be expected to cumulatively enhance binding properties when combined in the variable domains of the antibody construct. Some of these changes may diminish stability, but through the use of a hyperstabilized starting construct generated by combining multiple stabilizing amino acid changes, this loss of stability may be partially compensated. If the variant of improved binding properties exhibits unsatisfactory stability, additional stabilizing variations may be introduced by the methods described herein.

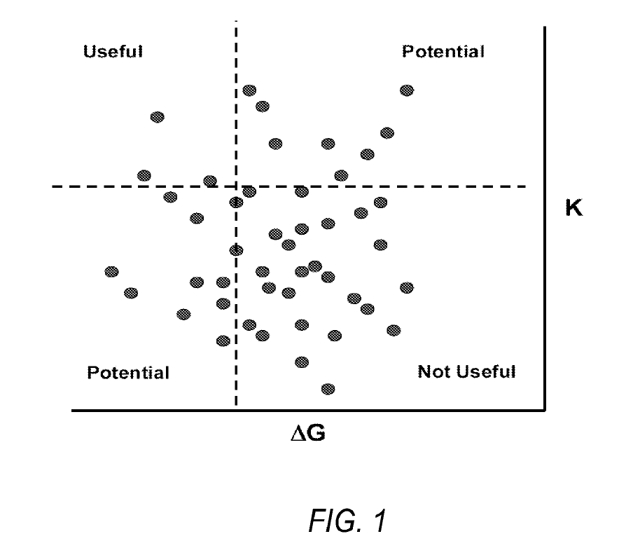

Usefulness of an antibody depends on the affinity for a specific antigen and the stability. As shown in FIG. 1, antibodies with lower affinity and stability are generally not useful or are of limited utility. Antibodies with higher affinity but lower stability have the potential that could be realized only if the stability is significantly improved. Antibodies with higher stability but lower affinity also have the potential that could be realized only if the affinity is improved. Finally, antibodies with higher affinity and stability are ideal for most applications. While there are number of methods available for increasing the affinity, there are very limited methods that can be used to increase the stability. Most of the methods currently used for increasing stability are random, costly, time consuming, and unpredictable. On the other hand, the method described herein is systematic, inexpensive, fast, and predictable.

Antibody light chains are overproduced when cells that generate antibodies become malignant as in the cancer multiple myeloma and other conditions. In some cases the light chains aggregate and may become the ultimate cause of death. Some of these aggregates are designated as amyloid, a fibril that is formed by the protein. Amyloid fibrils are found in other diseases and are produced by at least 20 different proteins. A major example is the amyloid that is the basis of plaques found in the brains of patients that die with Alzheimer's disease.

Amyloid formation by immunoglobulin light chains provided a unique clinical challenge. Due to the fact that the light chains produced by different patients invariably exhibit numerous amino acid variations, it was impossible to identify specific amino acid variations that could be considered the “cause” of amyloid formation, a fatal complication in 10 15% of patients with cancers of immunoglobulin-producing cells. Ultimately, the inventors demonstrated that the cause of fibril formation was the cumulative destabilizing effect of the naturally occurring mutations that are the basis of extreme diversity of binding properties by the immune system. Decreased stability of light chains is not a biological problem, unless the light chain is over produced as a result of malignancy of the cell producing it.

Because all human antibodies are constructed from a finite number of germline genes, the work required to significantly stabilize any antibody is not an open ended project even though amino acid replacements that improve the stability of one germline gene product will not necessarily have the same consequence in all germline products. Ultimately, most work required to stabilize a new antibody will be largely guided by the results of prior studies, with comparatively little new screening of amino acid variations required.

Melting temperature of engineered antibodies is an application criterion. For example, 65° C. melting temperature (Tₘ) is acceptable for engineered antibodies for therapeutic applications. However, diagnostic applications require engineered antibodies with Tₘ values of 65°-70° C., and for use as field-deployed biosensors engineered antibodies with Tₘ value as high as 80° C. will be required.

The melting temperatures of light chain and heavy chain variable domains in functional antibodies range from approximately 25° C. to 70° C. If the antibody producing B-cell combines a very unstable heavy chain variable domain with an equally unstable light chain variable it is probable that no functional antibody will result. However, it is not unusual to find antibodies in which one domain has good stability with melting temperatures ranging from approximately 50° C. to 70° C. while the other domain has marginal stability; i.e. melting temperatures between 25° C. and 40° C. Such antibodies are immunologically functional in that an extended serum half-life is not essential; the immune system continually produces the antibody as needed. However, antibodies in which one of the domains is significantly unstable have marginal biotechnological utility due to limitations that include production quantity, shelf-life, and range of applications.

As a consequence, the optimal and/or minimal melting temperatures for the domains in an antibody is a direct function of its intended application, such as therapeutics, diagnostics, or biosensors. Since several therapeutically useful antibodies are already in clinical use and hundreds are in drug discovery pipelines, it is evident that extreme levels of stability are not required. Therefore, it is reasonable to estimate that the minimal melting temperature of the domains of therapeutic antibodies is in the range 45° C.-50° C., given that physiological temperature is 37° C. An upper limit can be estimated as between 60-80° C. Many potential applications of antibodies in biosensors would require that the antibody tolerate elevated temperatures for a significant period of time. Exposure to realistic temperatures of 120° F. would be endured by antibodies composed of domains that have melting temperatures in excess of 80° C. (176° F.). Conventional applications of immunodiagnostics in well controlled clinical laboratory environments require a melting temperature range comparable to that of therapeutic antibodies. However, antibodies for which the melting temperatures of both variable domains is in the range of approximately 60° C.-80° C. should enable development of diagnostic applications in which refrigerated transport and storage of reagents is minimized. In each of the examples cited above, the upper limits specified are not intended to imply that stabilities that exceed the upper limit are disadvantageous. Rather, the upper limit represents a stability level at which additional experimentation to further increase the melting temperature would have little benefit for the specified application. As a result, the intended use of a given antibody defines the optimal stability of the variable domains as well as the effort/cost associated with meeting the specified melting temperature targets.

Selection of potential amino acid changes that might increase the melting temperature of the variable domains is based on a comparison of the amino acid sequence of the target protein to that of its homologs; i.e., proteins with which the variable domain has a common evolutionary relationship. In case of proteins for which the three-dimensional structure is not known, a requirement of at least 25% sequence identity virtually assures that the target protein and its apparent homologs have the same structure. In the case of an antibody variable, these homologs are represented by other antibody variable domains as well as many other proteins, such as T-cell receptors, in the immunoglobulin superfamily within which the same basic structure is found. However, low sequence identity increases the probability that the potential stabilizing effect of a particular amino acid change can only be recognized in the context of a second, complementary, change at another position in the protein. Contrariwise, homologs that have high sequence identity (>90%) clearly represent little information content. The preferable range of sequence identity for the homologs used to compile a roster of amino acid changes too be screened for their ability to increase melting temperature is 40-90%.

The strategy described herein might be termed “genetically” directed, and is distinct from all the prior approaches. The method described herein is based on screening of homologs of the molecules to evaluate amino acid variability at each position. Homologous proteins are related by conservative amino acid substitutions that have occurred since their original evolutionary divergence. Substitution of one amino acid side chain for another one within the same physicochemical group is a conservative substitution. Amino acids observed at each position in the homologous polypeptide represent an amino acid that is compatible with the three-dimensional structure. Criteria for recognition of homologs; i.e., proteins that evolved from the same evolutionary precursor and probably share similar three-dimensional structures, include that a statistically significant fraction of amino acids in a conservative alignment; i.e., minimal use of insertions or deletions in the sequences, are identical. Although distant homologs do not necessarily have similar 3-D structures, restriction of comparison to homologs with at least 25% sequence identity increases the likelihood that amino acids at corresponding positions play similar structural roles.

Most homologs (proteins with a common evolutionary progenitor) do not have statistically significant sequence identity. Use of a 25% identity cutoff allows confidence that the two sequences encode proteins that have the same fold despite amino acid variations that may enhance or impair stability. Of more significance than the overall sequence identity is the frequency at which certain amino acid changes are observed. For instance, if 80% of the sequences have arginine at position N, and there is one example of Ser, then it would probably be reasonable to ignore Ser and not bother to screen for the consequence of substituting it for Arg, essentially considering the appearance of Ser due to a fluke mutation or a sequencing error. However, if 15% of the sequences have Ser, then it would be useful to screen the consequences of that substitution.

The generation of a list of all known evolutionarily permitted ammo acid replacements substantially reduces the number of amino acid variations to be screened compared to the original 19^{N} alternatives. However, the total number of variations found in the database may be large. Indeed, the larger the number of variations observed, the higher the probability of successfully controlling the stability of the protein, albeit with a larger amount of potential experimental work, to screen the physicochemical consequences of the amino acid changes. Homologs obtained from organisms of similar environmental niches, or from animals of comparable physiological body temperatures, exhibit, on average, equivalent thermal stabilities. Thus, some of the amino acid changes may show little physicochemical effect. However, in sequences in which destabilizing amino acid changes are present there necessarily exist one or more stabilizing changes to compensate. During evolution, the stabilizing or destabilizing changes may occur in either order, although severely destabilizing alterations can probably only successfully take place in a fortuitously overstable variant of the protein.

Screening of amino acid variations is prioritized to minimize the experimental work necessary to achieve the stability goal, which may vary on a case-by-case basis, and is evaluated experimentally. Depending, on the application, original stability may need to be enhanced, reduced, or otherwise modified. For instance, the thermal stability of antibodies to be used in future field-deployable biosensors is required to be significantly higher than that required for therapeutic antibodies. Prioritization is based on the number of amino acid variations at each position in the polypeptide chain. The validity of the apparent prioritization is dependent upon the existence in the protein sequence database of a sufficient number of homologs to provide a representative sampling of evolutionary successes for the protein structure; i.e. on the order of 1000. The database contains >100,000 amino acid sequences for antibody variable domains which represent the most heavily sampled protein family.

Priority for experimental evaluation of the consequences of amino acid changes is based on (a) the amount of variation seen at each position, and (b) the structural location of the position; i.e., the possibility of amino acid changes interfering with function and, in the case of therapeutic antibodies, immunogenicity.

At positions at which no variability is observed, no mutations will be introduced in screening. It is assumed that no Observed variability implies an important structural or functional role for that position and the likelihood of finding, an enhancing replacement is small, but not impossible. However, the protein sequence database provides no guidance for choice of amino acid replacement; random screening is not included in our strategy for stabilization.

Screening of amino acid changes at positions where variations have been found is prioritized by the number of different amino acids observed. Changes of amino acids by site specific mutation are systematically assigned on the basis of positions having the lower number of variations given the highest priority. For instance, the first round of screening is undertaken with a list drawn from sites having two, three, or more alternative amino acids at a position in a protein until the experimental capacity was filled. “Experimental capacity” might be 20 mutations for a single technician working manually but can be multiples of 96 if the mutations are undertaken robotically. It is assumed that positions with the highest number of observed amino acid alternatives are positions of high tolerance of variation and that the probability of finding stabilizing or destabilizing variations decreases as the number of alternatives increases. At sites of high variation (>10 different amino acids), lower priority is given to amino acid changes that are likely to be structurally inappropriate; i.e., introduction of a charged residue into the hydrophobic core of the protein or introduction of hydrophobic residues to the exterior. In general, hydrophobic substitutions in the interior of the protein will be given higher priority in hypervariable positions.

Amino acid variations that are identified as changing stability in the single site mutational screening are combined in a single protein to achieve a cumulative change in the stability. Amino acid changes that involve functional sites on the protein are not used. Amino acid changes that involve independent alterations in structural properties are likely to be cumulative; amino acid changes that introduce competitive interactions, such as two side chains making hydrogen bonds to the same atom, are unlikely to be fully cumulative.

The method described herein stabilizes antibodies in a timely, cost-effective, and predictable manner. The method is suitable for the stabilization of any antibody and can generally be accomplished within about three months or less after obtaining the necessary genetic information. This time period systematically decreases as a database of enhancing amino acid substitutions grows.

In an embodiment, stability of a protein engineered by the methods described resulted in a 2,000 fold increase in stability compared to its original counterpart.

In another example, the structural basis of amyloid fibril formation by human antibody light chains, a fatal complication of the cancer, multiple myeloma, was identified. A human light chain variable domain was engineered by a combination of seven stabilizing amino acid substitutions. This construct was more than 1,000,000 times more stable than the original variable domain in terms of the improved ratio of normal structure to unfold.

Examples of successful control of stability include: anti-laminin antibodies: 1000-fold improvement in stability without compromising its binding ability to laminin; anti-botulinum neurotoxin (BoNT) and anti-anthrax spore antibodies: light chains were highly unstable; succeeded in increasing Tₘ to 50° C.; combination of stabilizing amino acid changes in anti-BoNT antibodies resulted in Tₘ of 65° C.

### Methods

Using standard algorithms such as FASTA [Lipman and Pearson (1988) Improved tools for biological sequence comparison. Proc. Natl. Acad. Sci. USA 85: 2444], BLAST or Psi-BLAST [Altschul S. F., Madden T. L., Schaffer (Altschul et al, 1990) A. A, Zhang J., Zhang Z., Miller W., and Lipman D. J. (1997) Gapped BLAST and PSI-BLAST: a new generation of protein database search programs. Nucl. Acids Res. 25: 3389-3402], developed to evaluate amino acid sequence similarity between proteins, a multiple sequence alignment of probable homologs is compiled. Table 3 shows an example. Relative to BLAST, Psi-BLAST permits searches to extend to more distant evolutionary relationships. Homologs are proteins having a common evolutionary descent. Alignments are optimized for placement of insertions and deletions (that compensate for differences in the length of amino acid chains) to assure compliance with locations that are consistent with the known three-dimensional structure of the protein. This information is not used by the algorithms cited in the previous paragraphs.

If the three-dimensional structure of the protein of interest is known, or the structure of one of its homologs is known, amino acid changes that introduce a charged amino acid to the core of the folded protein, or introduce hydrophobic amino acids to its exterior are eliminated.

Amino acid changes are systematically prioritized starting with positions of fewest alternatives and placing higher priority on amino acid changes in the interior of the protein. Numerous existing antibody structures provide extensive guidance for identification of interior and exterior amino acids. Generally, only about half of the approximately 120 amino acid positions found in antibody light and heavy chain variable domains represent high priority positions for amino acid substitutions.

Using well-established standard techniques of protein engineering [Raffen R., Dieckman Szpunar M., Wunschl C. Pokkuluri P. R. Dave P. Wilkins-Stevens P, Cai X., Schiffer M, and Stevens F. J. (1999) Physicochemical consequences of amino acid variations that contribute to fibril formation by immunoglobulin light chains. Protein Sci. 8: 509-517], site specific variants are created that incorporate the amino acid replacements.

Genes coding for the proteins corresponding to the original (target) sequence and variants in which single amino acid changes were incorporated, were cloned and expressed. Variants for which expression yields (level of protein production) were less than that seen in the original protein, were discarded, since these variants are likely to be destabilized. Variant protein constructs for which protein yield levels are comparable to or better than those observed in the original protein, were purified.

Stability of the original protein and variants was quantified. Stability may be measured in terms of thermodynamic equilibrium or by tolerance of elevated temperature, pH variation, or other challenges. The term “stability” refers to the ability of a protein to maintain its native conformation and function in response to changes in environmental factors such as temperature, pH, and ionic strength. However, it is important to note that two proteins may have similar thermodynamic stabilities (ratio of properly folded protein to unfolded protein) but differ, for example, in their response to temperature and pH.

Some amino acid changes may have negative consequences on the function of the protein. Such variants are not considered further. Amino acid variations that improve stability without functional consequence in the same domain are combined.

Stabilizing variations were iteratively combined until (a) the desired level of stabilization has been reached as determined by an appropriate method; i.e., unfolding in a chemical denaturant for thermodynamic stability, unfolding as a function of exposure to elevated temperature for thermal stability, preservation of function or fold upon changes of pH, etc., or (b) the pool of identified variations has been exhausted without reaching the stabilization goal, which is determined by the ultimate application of the protein. The desired level of stabilization will vary from case to case. For instance, antibodies that are to be used for therapeutic applications may be optimal with light chain and heavy chain melting temperatures of 65° C. Antibody-based biosensors to be used in benign environments such as airports or office buildings may perform adequately with melting temperatures of 75° C. whereas biosensors that are to perform under more extreme field conditions may require antibodies with melting temperatures of at least 85° C.

If the stabilization goal has not been achieved, steps are repeated.

Stabilized antibodies are the tested to assure affinity and kinetic properties that meet predetermined design specifications. Systematic amino acid changes can be used to identify replacements that improve affinity, kinetics, and specificity.

### Biosensor Development.

Antibodies are used extensively in the immunodiagnostics industry. However, each diagnostic test requires execution of a separate analysis in the clinical laboratory. There is extensive variation in the protocols used. An emerging concern raises a new challenge for the use of antibodies to cope with the threat of bioterrorism and biowarfare. In this instance, the nature of the threat will not be known a priori and could include anthrax, botulinum toxin, ricin, ebola virus as well as several other agents that can be used singly and in combination. A need exists for the means to test for all these agents simultaneously at the site of concern rather than back in the laboratory.

To date, three technical flaws render field-deployable multiplexed, antibody-based sensors unfeasible: (1) low stability limits temperature stresses tolerated by antibodies, (2) functional heterogeneity makes many assay protocols incompatible, and (3) requisite control proteins suffer the same stability issue as the capture antibodies.

These three flaws are correctable by the application of the methods described herein because: (1) stabilization allows non-laboratory antibody applications, (2) hyperstabilization allows modification of contact residues to improve binding characteristics while retaining adequate stability, and (3) stabilization of anti-idiotypic scFv constructs provides controls.

In summary, antibody stabilization capability is of direct relevance to conventional applications for immunodiagnostics and immunotherapeutics, but also creates new opportunities. These opportunities include, but are not limited to, biosensor development.

### EXAMPLES

### Example 1

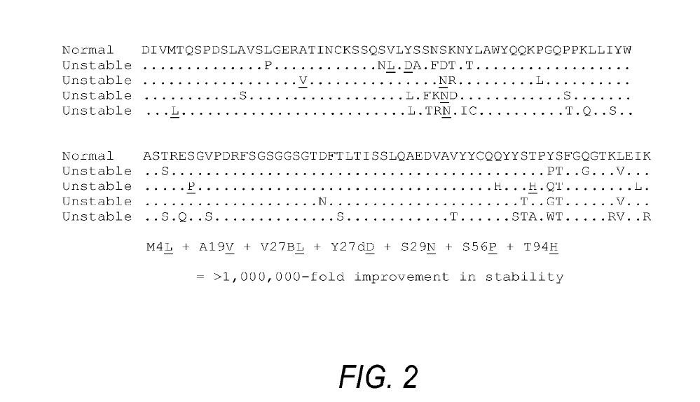

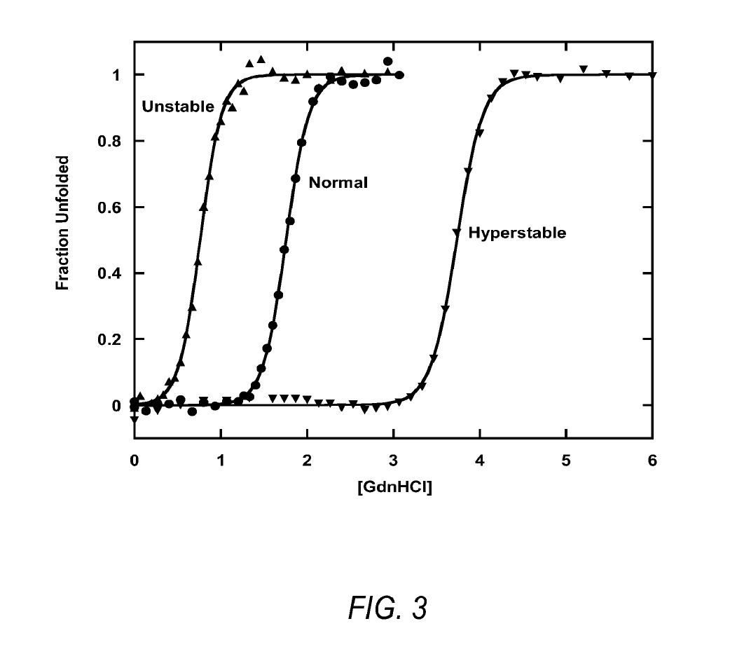

FIG. 2 shows an example of multiple alignment of amyloid light chain (human kappa-4) sequences from various myeloma patients. The top sequence is that of an amyloid light chain produced by a myeloma patient who experienced no clinical problems due to the protein. For the sake of convenience, it is considered as a “native” protein and therefore provides a baseline of normal stability. The other four sequences (only differences from the sequence of the normal protein are listed) are those of four amyloid forming kappa-4 light chains produced by different myeloma patients, encoded by the same germline gene as the normal protein. Taken separately, all of the sequences have significantly reduced stability. However, all incorporated some variations that improved stability, and those variations are indicated by underlining. When seven of these changes were combined, the result was a kappa-4 chain that had about 1,000,000-fold higher thermodynamic stability than the normal, and 1,000,000,000-fold higher than the least stable amyloid light chain (FIG. 3). Thermodynamic stability refers to the equilibrium between native and unfolded forms of the protein at a given temperature.

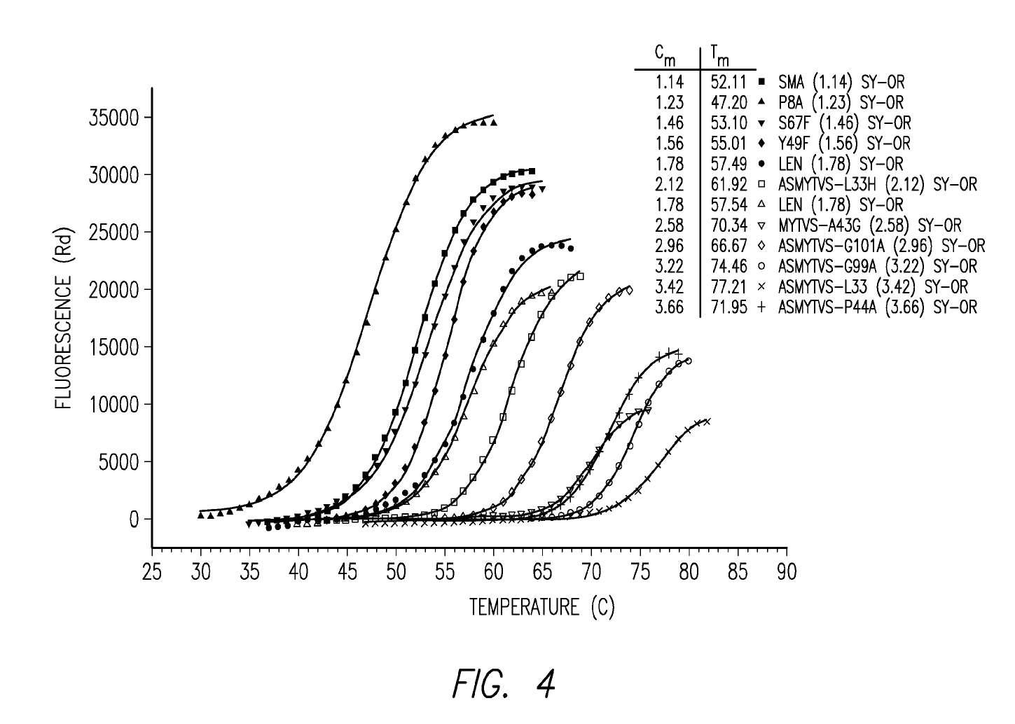

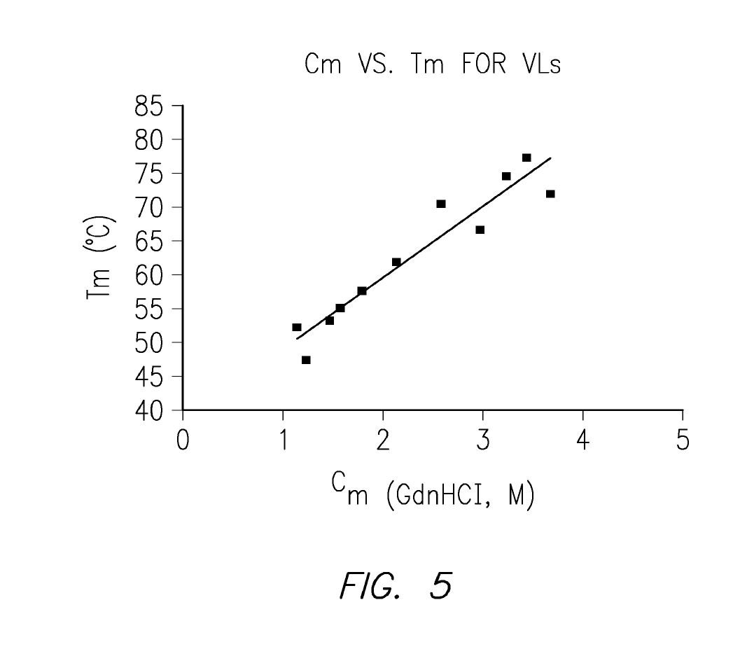

A panel of mutants were also subjected to the determination of thermal stability, which indicates the endurance of a protein to elevated temperature. Thermal stability is determined by measuring the “melting temperature” (Tm), which is defined as the temperature at which half of the molecules are denatured. Thermal denaturation curves of the native protein and mutants show that several of the mutant constructs are more resistant to thermal denaturation than the native protein (FIG. 4). The “native” protein, designated Len (an abbreviation of the patient's surname) in Raffen et al. (1999) was produced in large quantities by the patient without clinical complication. Thus, this protein is a highly suitable control for experimental studies of the origin of pathology. As shown in FIG. 5, there is a good correlation between the two measures of stability (thermal stability as expressed by Tm values and thermodynamic stability as expressed by Cₘ values) although, as expected because of different contributions of various amino acids to enthalpy and entropy effects, the correlation is not absolute.

### Example 2

As shown in Table 1, eleven amino acid changes were proposed for screening changes in stability for a human kappa-1 antibody light chain variable domain. Four of the changes were found to increase stability (highlighted in bold). The four amino acid changes were dispersed within the structure of the protein; thus, it was anticipated that the stability changes would be additive when combined within a single domain.

Replacement of alanine by valine at position 13, leucine by isoleucine at position 47, phenylalanine by leucine at position 73, and leucine by valine at position 78 confirmed this prediction, resulting in a 2000-fold improvement in the thermodynamic stability of the protein. The modified variable domain required an increased denaturant concentration of approximately 1 mole to achieve 50% unfolding, indicative of increased stability corresponding to a change in free energy of folding of −5.0 kcal/mole. The thermodynamic equilibrium constant of the original domain was 6×10⁴; i.e., the ratio of correctly folded to unfolded forms of the protein was 6×10⁴. In the variant that incorporated the four amino acid changes, the ratio increased to 1.5×10⁸, corresponding to the 2000-fold increase that was predicted. The results illustrate that thermodynamic stability can be systematically improved by combining amino acid changes that were identified as stabilizing by single-site mutagenesis. The amino acid replacements that decreased stability are also informative because they may indicate destabilizing amino acids in variable domains of other antibodies. The above experiment was completed in six weeks.

### Example 3

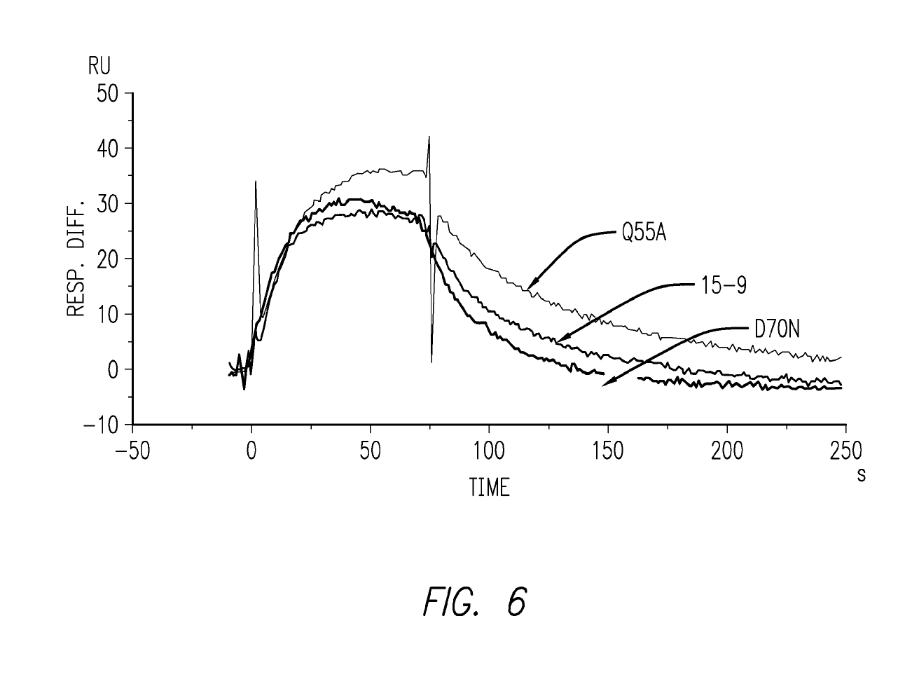

An antibody must be stabilized without impairing function. To examine this, two anti-laminin scFv constructs were modified with different amino acid replacements, and 1000-fold improvement in stability was achieved. The stabilizing mutations were combined in a single domain, resulting in an approximate ten-fold increase in yield compared to that obtained with the original anti-laminin construct. Binding of the mutants to laminin was monitored using Biacore instrument. As shown in FIG. 6, there was no significant difference in binding to laminin between the native protein and the mutants. This indicates that improved stability can be achieved without sacrificing performance. Alternative selection of amino acid substitutions and/or the availability of more than one useful antibody/scFv for the target of interest will minimize this occurrence.

### Example 4

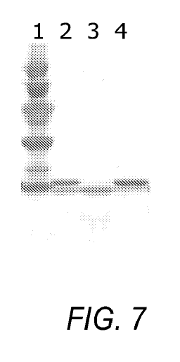

For therapeutic applications of antibodies, the ability of the protein to survive in the body has a direct relationship to clinical efficacy. Human serum contains proteases, enzymes that breakdown other proteins. Proteins that are unfolded expose more vulnerable sites to proteases, and are destroyed more rapidly. Improved thermal and thermodynamic stability enhances resistance to proteases as illustrated in FIG. 7.

From left to right are depicted molecular weight standards, a wild-type heavy chain variable domain (VH2-wt), the domain destabilized by a single amino acid change (VH2-6), and the domain stabilized by a single amino acid change (VH2-15) after incubation with trypsin at a ratio of 1:20 enzyme to protein. As shown, the wild-type form reveals significant production of a fragment (lower band). The destabilized form is completely converted to the smaller fragment, while the stabilized variant shows little fragmentation.

### Example 5

Table 2 provides a few examples of candidate antibodies for stabilization by methods described herein. These examples were taken from the protein structural database and include antibodies of potential therapeutic and diagnostic application. Numerous additional candidates of antibodies that have potential commercial importance can be found in the databases of patented protein sequences.

### Example 6

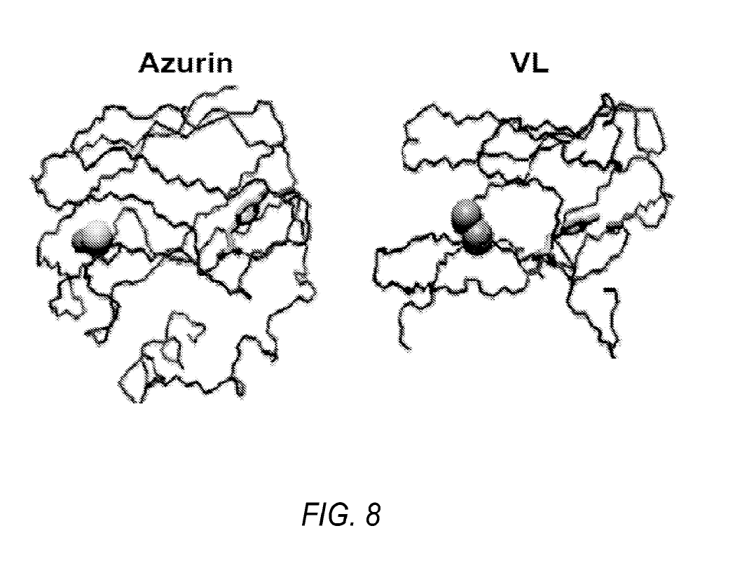

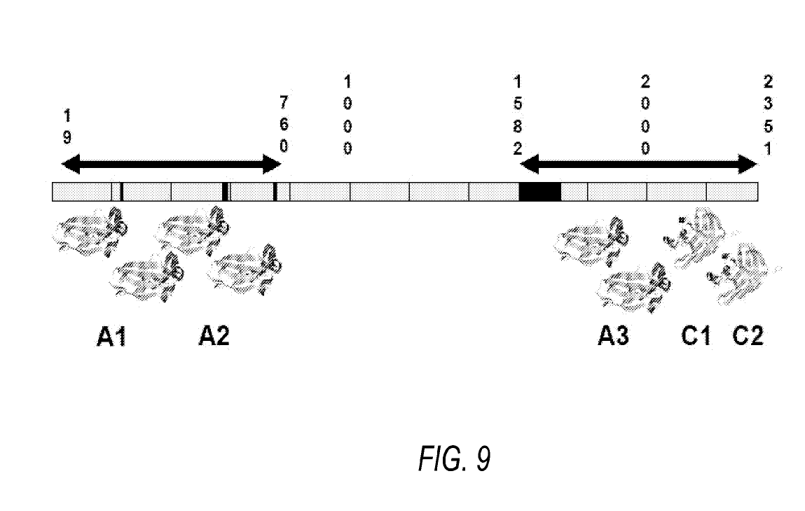

Many proteins with immunoglobulin-like structures are of therapeutic relevance and are candidates for stability enhancement. For instance, the protein, Factor VIII, is a major component of the blood coagulation pathway. Numerous mutations or polymorphic amino acid variations result in hemophilia. As a result, production of Factor VIII, or derivatives of it, is a major pharmaceutical effort since hemophilia patients require replenishment of Factor VIII on an on-going basis. Structural studies of this protein have revealed that the functionally critical portions of the molecule consist of domains related to the proteins cupredoxin and lactadherin (FIG. 8). Cupredoxin and lactadherin domains may also have an evolutionary relationship to immunoglobulins. By analogy to the facility with which the stability of antibody variable domains can be improved, the A1, A2, A3, C1 and C2 domains of Factor VIII are suitable for significant stability enhancement (FIG. 9).

Candidate amino acid changes for stabilization of the eight immunoglobulin-like domains of human coagulation protein Factor VIII (gil182803) are described below (Table 3). The amino acid sequences are presented on the basis of the conventional nomenclature for Factor VIII except that domains A1, A2, and A3 are subdivided (e.g., A1a and A1b) to indicate each of the two cupredoxin domains that are present. The top line in each Table provides the amino acid sequence of the domain found in human Factor VIII. Below are tabulated alternative amino acids observed in at least one homolog produced by approximately 35 other species as limited by an approximate cutoff of about 50% sequence identity. In some cases, the amino acid found in the human protein was not the most common. Additional candidate changes can be obtained by lowering the identity criterion.

Previously all protein stabilization projects were considered high-risk, potentially long-term, expensive undertakings with no assurance of success. Experimental strategy was usually “guess-and-check.” Current results suggest that at least some classes of protein are capable of being efficiently modified to improve stability.

### Materials and Methods

scFVs

Synthetic DNA encoding Bot1 or Anx1 scFVs was obtained from Blue Heron Biotechnology. The coding sequences were optimized for expression in E. coli and contained terminal restriction sites for subcloning into the expression vector pET22b. Individual VH and VL domains from each scFV were also amplified by PCR using primers containing restriction sites for subcloning into a modified version of the E. coli expression vector pASK40 [Skerra A., Pfitzinger I. and Pluckthun A. (1991) The functional expression of antibody Fv fragments in Escherichia coli: improved vectors and a generally applicable purification technique. Biotechnology (NY) 9:273-8.] The modifications to pASK40 included addition of restriction sites (to aid in subcloning) and addition of residues encoding 6 C-terminal histidine residues the pASK40 vector (for purification by immobilized metal affinity chromatography).

### Analysis of VL and VH Stability by Thermal Denaturation

Relative stability of VL and VH domains was analyzed by thermal denaturation of the proteins in the presence of the protein dye SYPRO Orange. [Niesen F. H., Berglund H. and Vedadi M. (2007) The use of differential scanning fluorimetry to detect ligand interactions that promote protein stability. Nat Protoc 2:2212-21.] Briefly, 40 μl of 10-20 μM protein sample in PBS and containing 5×SYPRO Orange was heated from 25 to 90° C. in 1° C. increments in a MX4000 qPCR system (Stratagene) with excitation at 492 nm and emission at 580 nm. Protein unfolding was detected as an increase in fluorescence upon binding of the dye SYPRO Orange to the denatured protein. The transition midpoint was determined by nonlinear least squares curve fit of the data to the Boltzman equation using the program Prism 4 (GraphPad Software). (Altschul, et al., 1997).

### Site Directed Mutagenesis

This technique as described in Raffen et al. (1999) was used to examine the consequences of individual amino acid substitutions in proteins.

### Database Searches

Psi-BLAST was used for all sequence searches through the NCBI website (www.ncbi.nlm.nih.gov). (Altschul, 1997) Default parameters were used with the exception that the number of alignments and descriptions was set to 5000. Degree of sequence identity and expectation values were not used as parameters of valid alignments; more than 50% alignment of the query sequence to a putative match was required. Psi-BLAST iterations were continued until convergence or the degree of sequence identity of the highest scoring match fell below 25%. Alignments were used as provided by Psi-BLAST with the exception of minor adjustments made as suggested by consensus of multiple alignments. Alignments of sequences identified by Psi-BLAST as possible homologs were also aligned by Profile Multile Alignment with predicated Local Structure (PROMALS) [Pei J. and Grishin N. V. (2007). PROMALS: towards accurate multiple sequence alignments of distantly related proteins. Bioinformatics 23: 802-808], which also includes information from secondary structure predictions and Hidden Markov models. The PROMALS system, essentially uses secondary structure prediction and profile-profile Hidden Markov Model approaches to facilitate alignments of sequences with low levels of identity.

All references, including publications, patent applications, and patents, cited herein are hereby incorporated by reference for materials and methods used herein to the same extent as if each reference were individually and specifically indicated to be incorporated by reference.

TABLE 1
Stabilization of a human κ-1 light chain

| Construct | Cm |
|---|---|
| Q37L | 0.73 |
| I21L | 1.06 |
| R18P | 1.07 |
| A13L | 1.20 |
| V58I | 1.29 |
| Base | 1.33 |
| L78I | 1.33 |
| L11V | 1.35 |
| L47I | 1.47 |
| A13V | 1.52 |
| F73L | 1.66 |
| L78V | 1.74 |

TABLE 2
Suitable Antibodies

| (Antibodies) | Protein Data | |
|---|---|---|
| Proteins | Base Identifier | Reference |
| Anti-Dengue | 2R69 | Lok et al, 2008 |
| Anti-Ebola | 2QHR | Lee et al., 2008 |
| Anti-HIV | 2B0S | Stanfield et al, 2006 |
| Anti-Lyme Disease | 1FJ1 | Ding et al., 2000 |
| Anti-Malaria | 1OB1 | Pizzaro et al., 2003 |
| Anti-Malaria | 2J4W | Igonet et al., 2007 |
| Anti-Malaria | 2Q8B | Coley et al, 2007 |
| Anti-Morphine | 1Q0Y | Pozharski et al., 2004 |
| Anti-Neisseria Meningitidis | 1QKZ | Derrick et al,, 1999 |
| Anti-SARS | 2DD8 | Prabakaran et al,, 2006 |
| Anti-West Nile Virus | 1ZTX | Nybakken et al, 2005 |

TABLE 3A
Table 3A: positions 20-205; the most distant homolog is from Gallus
gallus and exhibited 57% amino acid identity. Table 3A discloses SEQ ID
NOS: 1-7, respectively, in order of appearance.

| | | |
|---|---|---|
| 20 | ATRRYYLGAVELSWDYMQSDLGELPVDAREPPRVPKSFPFNTSVVYKKTLFVEFTVHLFN | 79 |
| | .I.K..I.T..TP.N.TSGEASAQHMGTSGSSTMSRTL.LSNHIMHR.AV...YKDDF.S | |
| | ............A...IH.G.RVARA.S..LAGA.GA...TP..T.T.......M.QS.H | |
| | ................R....LQ..T.V.....T.TP...GA..L.S.......L.R..T | |
| | ................V........S.........A........R...........A... | |
| | ............................................................ | |
| | ............................................................ | |
| 80 | IAKPRPPWMGLLGPTIQAEVYDTVVITLKNMASHPVSLHAVGVSYWKASEGAEYDDQ | 136 |
| | VPR.K.L.........RT..H....VVF..L..RAYN.....M.F..S...DA.E.E | |
| | T.....A.........W...................................G...H | |
| | ......................................................... | |
| | ......................................................... | |
| | ......................................................... | |
| 137 | TSQREKEDDKVFPGGSHTYVWQVLKENGPMASDPLCLTYSYLSHVDLVKDLNSGLIGALLVCREGSLAK | 205 |
| | STKS.QDG.N.I..ETY..A.HIQGDS..TDG.SP...HA.F.N.NP.R.T....V....I.KP.R.SR | |
| | ..PT.....R.L..K.Q..I.I..Q.Q.......Q......M....S.N................T.T. | |
| | ...K.......D.........E...................S.........................V. | |
| | ...M................................................................. | |
| | ...A................................................................. | |
| | ...P................................................................. | |

TABLE 3B
Table 3B: positions 206-390; Gallus gallus; 57%. Table 3B discloses
SEQ ID NOS: 8-14, respectively, in order of appearance.

| | | |
|---|---|---|
| 206 | EKTQTLHKFILLFAVFDEGKSWHSETKNSLMQDRDAASARAWPKMHTVNGYVNRSLPGLIGC | 267 |
| | DRARIPYTLVM........RT.YTARPEPYIRAMASGATQDRHEL..I...T.GT....TV. | |
| | .T..M.QE...........E...G.AND.WTHSGTP.PKEPO.RV......I.S.....VL. | |
| | .G..N.PQ...............A...A.KA.T..Y...K.S.T.................. | |
| | ....H..H...................G.P................................ | |
| | ...........................P.................................. | |
| | .............................................................. | |
| 268 | HRKSVYWHVIGMGTTPEVHSIFLEGHTFLVRNHRQASLEISPITFLTAQTLLMDLGQFLLFC | 329 |
| | RKRQIH...MAV..S.DI...L..S...F..H..LT....P.LSL...E.VPITT.R.RN.. | |
| | .GTL.......S..G......F......S..S...S......M.Y.....F.G.A.H..V.. | |
| | ...............................G..........A.......W.....S..... | |
| | ..................................................M........... | |
| | .............................................................. | |
| | .............................................................. | |
| 330 | HISSHQHDGMEAYVKVDSCPEEPQLRMKNNEEAEDYDDDLTDSEMDVVRFDDDNSPSFIQI | 390 |
| | QMP..KQG....HIE.EK.ADAS.WQKHAHSDEKY.ENGFA..D..MFTL.G.SPAFYV.V | |
| | .....H.A....S.R..N....I.GKRGRS..MPE..EN.Y .....RSW.Y.DVS..... | |
| | .....R......V.Q..A......V...QD..T.......L..........P.AA...... | |
| | ..............H..E......Q....P............................... | |
| | ............................................................. | |
| | ............................................................. | |

TABLE 3C
Table 3C: positions 391-575; Gallus gallus; 58%. Table 3C discloses
SEQ ID NOS: 15-20, respectively, in order of appearance.

| | | |
|---|---|---|
| 391 | RSVAKKHPKTWVHYIAAEEEDWDYAPLVLAPDDRSYKSQYLNNGPQRIGRKYKKVRFMAYTD | 452 |
| | ..A...Y.VA.I...SVQ.D..E...SEATSSNGGHQNLY.SSHLHQL.K....AMYIE.E. | |
| | ..F...D..M.P.......G......AAPSLNATHLRRR..KR...GG.S.......V.... | |
| | ...........T.......V......VK.PSE...SS....GG................... | |
| | ...................M...........L...T.....EA................... | |
| | .............................................................. | |
| 453 | ETFKTREAIQHESGILGPLLYGEVGDTLLIIFKNQASRPYNIYPHGITDVRPLYSRRLPKGV | 514 |
| | ...TK.KPMPYGA.L...V.K.....SFK.V.RKK...A...H...LNY.SAVHTQKPSR.M | |
| | ...H...VTSR.Q.............Q.......R............RS.T.Y.AG..LQ.I | |
| | .......TQ.S.......................L...............G...PM.....W | |
| | .......KS.P.......................................N..........K | |
| | .......S...................................................... | |
| 515 | KHLKDFPILPGEIFKYKWTVIVEDGPTKSDPRCLTRYYSSFVNMERDLASGLIGPLLICYK | 575 |
| | .DV.HM..H..QT.T.R.KLAA.E..ARA.AP.V..F.A.SIDLDK.T...........C. | |
| | ..I..L..R...S.....DI.L.....Q..........Y.A..PQQ.............R. | |
| | .....V..A.........S..T.....................AV..............F. | |
| | .....I....................................................... | |
| | ............................................................. | |

TABLE 3D
Table 3D: positions 576-759; Gallus gallus; 69%. Table 3D discloses
SEQ ID NOS: 21-27, respectively, in order of appearance.

| | | |
|---|---|---|
| 576 | ESVDQRGNQIMSDKRNVILFSVFDENRSWYLTENIQRFLPNPAGVQLEDPEFQASNIMHSIN | 637 |
| | KTM......M...EMRLL...IL...Q...IA.DMR.LSLSATVLHPQ.SG.EL..L.YT.. | |
| | .............D...V.............S......CTEEDKTDKH....YT..V..... | |
| | ...............................P........D..H.................. | |
| | ...............................E...........E.................. | |
| | .............................................................. | |
| | .............................................................. | |
| 638 | GYVFDSLQLSVCLHEVAYWYILSIGAQTDFLSVFFSGYTFKRKMVYEDTLTLFPFSGEIFM | 699 |
| | .F..NH.E.TA.....TA.HV..V...S....I....H..E.RL.FA.V..V..H..V.Y. | |
| | .....G...RL.....V.......................R..A.S............... | |
| | .....A.....................................N................. | |
| | .....Q....................................................... | |
| | ............................................................. | |
| | ............................................................. | |
| 700 | SMENPGLWILGCHNSDFRNRGMTALLKVSSCDKNTGDYYEDSYEDISAYLLSKNNAIEPR | 759 |
| | TYDKL.V.VI..R.PEL.KS..S.KFT.YT.NRDID..SDNT.DGMPTHPVNEKHGVK.. | |
| | ......T.A.........E...H.....AQ.PESVS...GEI..EVIIS..AGDSILQ.. | |
| | ........N.........D.........T...LG.....L.Y...PLGF..RN..Y.... | |
| | ........T.......................................P......F.... | |
| | ................................................Q........... | |
| | ................................................D........... | |

TABLE 3E
Table 3E: positions 1667-1853; Gallus gallus; 53%. Table 3E discloses
SEQ ID NOS: 28-34, respectively, in order of appearance.

| | | |
|---|---|---|
| 1667 | REITRTTLQSDQEEIDYDDTISVEMKKEDFDIYDEDENQSPRSFQKKTRHYFIAAVERLW | 1726 |
| | .Q.PLSVVKPEEDKTE...IFTTDTTRQ.....GDEII.DL.N..QRI.Q.....M.QI. | |
| | .K.SV.AP.RQSNAFY...AV.I.I.G......S.YAK.G.....GQ..........E.. | |
| | ...I...I..H..NM....YS.S...I........N.G...................V.. | |
| | .......F..................P..........H...................... | |
| | ............................................................ | |
| | ............................................................ | |
| 1727 | DYGMSSSPHVLRNRAQSGSVPQFKKVVFOEFTDGSFTOPLYRGELNEHLGLLGPYIRAEV | 1786 |
| | E..LGRPLYA.GDSVWNDDAQRYR..I.R.YA....S.RSH....DK...I...C....I | |
| | ...VRK..RI.KATYRRKNFA..........L.......VM.....A............. | |
| | ...NQT..GF....G...E.R..................A.................... | |
| | .....E............P......................................... | |
| | .....V...................................................... | |
| | ............................................................ | |
| 1787 | EDNIMVTFRNQASRPYSFYSSLISYEEDQRQGAEPRKNFVKPNETKTYFWKVQHHMAPTKDEFDCKA | 1853 |
| | ..T.V.K.K.E....F..H...VG.KDAESG.EG..QK..N.Q.VEV.S.RERPQ....ER...... | |
| | ..........M...........Q..P..DGH.GA..RT..Q....RI.....LS.....DG...... | |
| | ..........L..................L......H...R.....E............MQ...... | |
| | ....................................S.............................. | |
| | ................................................................... | |
| | ................................................................... | |

TABLE 3F
Table 3F: positions 1854-2039; Taktfugu rubripes; 52%. Table 3F discloses
SEQ ID NOS: 35-43, respectively, in order of appearance

| | | |
|---|---|---|
| 1854 | WAYESDVDLEKDVHSGLIGPLLVCHTNTLNPAHGRQVIVQEFALFFTIFDETKSWYFTENME | 1915 |
| | ...S.N..KDR.MN...V...II.RAGV.SASRKTPESIR....VLAV........CP..LK | |
| | ...L.T......L........V..KSS..HSGFA..LA......L.HT........MA..VR | |
| | ...Y.....................R....TRLH..PD...................E...D | |
| | .........................P.................................... | |
| | .............................................................. | |
| | .............................................................. | |
| | .............................................................. | |
| | .............................................................. | |
| 1916 | RNCRAPCNIQMEDPTFKENYRFHAINGYIMDTLPGLVMAQDQRIRWYLLSMGSNENIHSI | 1975 |
| | .K.KT.RHVTIDN.ALRQEFS.R.V...VKES.L..LV.ENRKV..H..NVCNTGDTQAV | |
| | .Y.AP..GLPKG..HWHRSHK........G.A........G..T........DP.HA... | |
| | ........FNLQ..DY.I.N.........A..........Q............D.EY... | |
| | ........T.R...W.........................H................... | |
| | ........A................................................... | |
| | ............................................................ | |
| | ............................................................ | |
| | ............................................................ | |
| 1976 | HFSGHVFTVRKKEEYKMALYNLYPGVFETVEMLPSKAGIWRVECLIGEHLHAGMSTLFLVYSNK | 2039 |
| | ..HAQMVSIHTTQ.HRTGVC..F..I.G.L..Q.PQV.T.QI.SKV..SQQ...RATL...NKE | |
| | ....LS....DSG..............T....I..R....L...T.....R.....K.....TQ | |
| | .....P....AGK...................R..H..............L............R | |
| | ...................................T............................ | |
| | ................................................................ | |
| | ................................................................ | |
| | ................................................................ | |

TABLE 3G
Table 3G: positions 2040-2189; Dartio rerio; 52%. Table 3G disclosers
SEQ ID NOS: 44-51, respectively, in order of appearance.

| | | |
|---|---|---|
| 2040 | CQTPLGMASGHIRDFQITASGQYGQWAPKLARLHYSGSINAWSTKEPFSWIKVD | 2093 |
| | .NIAI..Q.RR.QNS.....DHH.L.T.N....NHA..V...MAQDAHP.LQ.. | |
| | ..A.......S.A.P......YE.P.K.Q....ENT.......GGSGA...... | |
| | ..V.......Y.T.K.........D...E.....S.........S..N...... | |
| | ..N.......Q.......................L................... | |
| | ..Q.......W........................................... | |
| | ...................................................... | |
| | ...................................................... | |
| 2094 | LLAPMIIHGIKTQGARQKFSSLYISQFIIMYSLDGKKWQTYRGNSTGTLMVFFGNVDSSG | 2153 |
| | ..TLV.V.S.M....KHQL.EFFVILYTVRH.N.QNN.HS.Q..T.SSQYM.NA.M.ATT | |
| | ..E.T.L...E.....TR.....T...S.F.....RQ.LK.K....KAY.T......G.R | |
| | ..H.K.....Q .....S...........S.....ST.KV.......P............ | |
| | ...................................QR.S..................... | |
| | ............................................................ | |
| | ............................................................ | |
| | ............................................................ | |
| 2154 | IKHNIFNPPIIARYIRLHPTHYSIRSTLRMELMGCD | 2189 |
| | VRA.T.D...VGQ..CVQ.STSQGQTA..I..L... | |
| | ..E.S.S.........IN.L.A.K.P......I... | |
| | ....R................P.............. | |
| | ....V................F.............. | |
| | ....L............................... | |
| | .................................... | |
| | .................................... | |

TABLE 3H
Table 3H: positions 2190-2351; Takifugu rubripes; 49%. Table 3H discloses
SEQ ID NOS: 52-59, respectively, in order of appearance.

| | | |
|---|---|---|
| 2190 | LNSCSMPLGMESKAISDAQITASSYFTHMFATWSPSKARLHLQGRSNAWRPQVNNPKEWLQVD | 2252 |
| | FA...I...LQNGV.A.TRVS...HLNSILTA.P.AQ...NQR.KT.....RGSDAEQ..... | |
| | .....A.....DRW.P.SSFV...SKSTT.SN.A..L...R.E.GA.....EAGS.T...... | |
| | .....L.......G...Q.......SAGA.RS.T..........S......KE.R.H...... | |
| | .............R...E.......YW.L.......................T.......... | |
| | ....................................................N.......... | |
| | ............................................................... | |
| | ............................................................... | |
| 2253 | FQKTMKVTGVITQGVKSLLTSMYVKEFLISSSQDGHQWTLFFQNGKVKVFQGNQDSFT | 2310 |
| | LRRLVRI.AII.K.ARAIFIN.F.TK.AVTT.R..RH.SPILYDDHT.IIKA.K.ASS | |
| | .GAVK.....V...T.HV.SA.M.R..SL.I....VN..QVRHKSGL...R..R.HTE | |
| | .E..................K..............QR..H..EG.RE......N.YD. | |
| | ....................................A..F.................. | |
| | .......................................R.................. | |
| | .......................................S.................. | |
| | .......................................................... | |
| 2311 | PVVNSLDPPLLTRYLRIHPQSWVHQIALRMEVLGCEAQDLY | 2351 |
| | TMMSCVES.RVA.EV.L..TIHGQH....I.I...DT.EQ. | |
| | EARLRFHA..EG..I....KV.ERD....L.F......QE. | |
| | ..LTA..............WG.A......V........... | |
| | ....P..............R..H.................. | |
| | ....I..............L..I.................. | |
| | ......................................... | |
| | ........................................ | |

TABLE 4
Therapeutic applications of antibodies

| Brand Name* | | |
|---|---|---|
| ** | Company | Application |
| [symbol] | Genentech/ | Colorectal carcinoma |
| | BioOncology | |
| Bexxar | Corixa/GSK | Non-Hodgkins lymphoma |
| Campath | Ilex Oncology/ | Chronic lymphocytic leukemia |
| | Millenium/Berlex | |
| [symbol] | ImClone/BMS | Colorectal carcinoma |
| [symbol] | Genentech | Metastatic breast cancer |
| [symbol] | CAT/Abbott | Rheumatoid arthritis |
| [symbol] | Genentech | Macular degeneration |
| Mylotarg | Celltech/Wyeth | Acute myeloid leukemia |
| Orthoclone OKT | Ortho Biotech | Acute kidney transplant rejection |
| Raptiva | Xoma/Genentech | Psoriasis |
| [symbol] | Centocor | Rheumatoid arthritis & Crohn's |
| | | disease |
| ReoPro | Centocor/Lilly | Blood clot related complications |
| [symbol] | IDEC/Genentech | Non-Hodgkin's lymphoma |
| Simulect | Novartis | Acute kidney transplant rejection |
| [symbol] | MedImmune | RSV infection |
| Tysabri | Biogen Idec | Multiple Sclerosis |
| Xolair | Genentech/ | Allergy |
| | Novartis/Tanox | |
| Zenapax | Roche | Acute kidney transplant rejection |

*Blockbusters in bold/italics, Compiled from Wang et al (2007) J. Pharm. Sci. 96: 1-26
** All registered trademarks

TABLE 5
Serum half-life of commercial antibodies

| Brand Name* | | Serum half-life |
|---|---|---|
| ** | Company | (days) |
| Amevive | Astellas | 11.3 |
| Bexxar | Corixa/GSK | 2.7-2.8 |
| Campath | Ilex Oncology/Millenium/Berlex | 12 |
| Erbitux | ImClone/BMS | 4.8 |
| Herceptin | Genentech | 2.7-10 |
| Humira | CAT/Abbott | 14.7-19.3 |
| Mylotarg | Celltech/Wyeth | 1.9-2.5 |
| Orthoclone OKT | Ortho Biotech | 0.75 |
| Remicade | Centocor | 9.5 |
| ReoPro | Centocor/Lilly | 0.29 |
| Rituxan | IDEC/Genentech | 9.4 |
| Simulect | Novartis | 4.1 |
| Synagis | MedImmune | 19-27 |
| Xolair | Genentech/Novartis/Tanox | 20 |
| Zenapax | Roche | 20 |
| Zevalin | Biogen Idec | 1.1 |

*Bold: extremely short half-life; Italic: very short half-life Compiled from Lobo et al (2004) J. Pharm. Sci. 93: 2645-2668
** All registered trademarks

TABLE 6
One antibody: multiple products

| Preferred | |
|---|---|
| Stability | Application |
| Low | Imaging, Radiotherapy, Therapy* |
| Medium | Therapy |
| High | Diagnostics, Biosensors |

*Only for therapy of short duration

1-21. (canceled)

22. A method for improving stability of a target protein, comprising:
(a) compiling a multiple sequence alignment of amino acids of probable homologs of the target protein;
(b) optimizing alignments of the amino acids for placement of insertions an deletions of the protein to assure compliance with locations of the amino acids that are consistent with a known three dimensional structure of the target protein, wherein the optimizing includes eliminating amino acid changes that introduce charged amino acid to the core of the target protein or introduce hydrophobic amino acids to the exterior of the target protein;
(c) systematically prioritizing amino acid changes such that positions corresponding to (i) amino acid changes in the interior of the protein; and (ii) having the fewest alternatives as determined from step (a) are given the highest priority for performing amino acid changes;
(d) creating a site specific variant of the target protein that includes the amino acid changes as determined from step (c);
(e) purifying the variant of the target protein for which an expression yield of the variant of the target protein is comparable or better than an expression yield of the target protein;
(f) quantifying a stability of the variant of the target protein;
(g) determining if the stability of the variant of the target protein corresponds to a desired level of stability; and
(h) repeating steps (d) to (g) until the stability of the variant corresponds to the desired level of stability or a pool of identified variants of the target protein has been exhausted.

23. The method of claim 22, wherein the compiling is performed using an algorithm comprising at least one of FASTA, BLAST, Psi-BLAST and Gapped BLAST.

24. The method of claim 22, wherein the expression yield of the variant of the target protein are determined via cloning and expressing genes coding for the variants of the target protein.

25. The method of claim 22, further comprising:
excluding amino acid changes that have a negative impact on a function of the target protein.

26. The method of claim 25, further comprising:
combining amino acid changes that improve stability of the target protein without having any negative impact on the function within a same domain of the target protein.

27. The method of claim 22, wherein the target protein includes an antibody.

28. A modified kappa-4 immunoglobin light chain comprising a mutations of: methionine at position 4 replaced with leucine; alanine at position 19 replaced with valine, valine at position 27 replaced with leucine; tyrosine replaced at position 27 with aspartic acid, serine at position 29 replaced with asparagine, serine at position 56 replaced with proline, and threonine replaced at position 94 with histidine.

29. A modified kappa-1 immunoglobin light chain comprising a mutations of: alanine at position 13 replaced with valine, leucine at position 47 replaced with isoleucine; phenylalanine replaced at position 73 with leucine, leucine at position 78 replaced with valine.

30. The modified kappa 1 immunoglobin light chain of claim 29, having a thermodynamic constant of at least 1.5×10⁸.
